(19) United States
(12) Patent Application Publication
(10) Pub. No.: US 2017/0095547 A1
(43) Pub. Date: Apr. 6, 2017

(54) MALARIAL ANTIGENS DERIVED FROM SUBTILISIN-LIKE PROTEASE 2 AND VACCINES AND METHODS OF USE

(71) Applicant: THE JOHNS HOPKINS UNIVERSITY, Baltimore, MD (US)

(72) Inventors: Jürgen Bosch, Pikesville, MD (US); Ryan Smith, Elkridge, MD (US); Daisy Del Carmen Colon Lopez, Baltimore, MD (US)

(21) Appl. No.: 15/314,115

(22) PCT Filed: Jun. 2, 2015
(86) PCT No.: PCT/US2015/033717

§ 371 Date: Nov. 28, 2016

Related U.S. Application Data
(60) Provisional application No. 62/006,388, filed on Jun. 2, 2014.

(51) Int. Cl. A61K 39/015; C12N 9/50

(52) U.S. Cl. CPC A61K 39/015; C12N 9/50; C12Y 304/21; A61K 2039/55566

(57) ABSTRACT

The present invention provides immunogenic compositions comprising one or more subtilisin-like protease 2 antigens, functional fragments or homologs thereof, from various Plasmodium species, methods, vectors comprising the antigens, and methods of use for the treatment of malaria and related disease.

### REFERENCE TO RELATED APPLICATIONS

This application claims the benefit of U.S. Provisional Patent Application No. 62/006,388, filed on Jun. 2, 2014, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on May 29, 2014, is named P12845-01_ST25.txt and is 3,376 bytes in size.

### BACKGROUND OF THE INVENTION

Obligate intracellular parasites from the genus Plasmodium are the agents responsible for malaria, placing an estimated 3.4 billion people at risk of the disease throughout the world. Five species of Plasmodium parasites cause human malaria, yet the largest impacts to public health are primarily caused by Plasmodium falciparum in sub-Saharan Africa, leading to approximately one million deaths every year.

Malaria parasites undergo a complex life cycle in their mosquito and human hosts, which require Plasmodium parasites to invade and replicate in multiple cell types and host environments. To accomplish these developmental progressions, Plasmodium parasites utilize specific invasion ligands and proteases to facilitate host cell invasion. Merozoite invasion of red blood cells (RBCs) has been studied in the most detail, and involves a large repertoire of surface proteins that contribute to multiple invasion pathways. Similarly, recent evidence suggests that ookinete invasion of the mosquito midgut may also involve multiple surface proteins and invasion pathways. While both merozoite invasion of the RBC and ookinete invasion of the midgut are rapid, these stages have attracted recent attention as targets for an asexual or transmission-blocking vaccines.

As a shared component of merozoite and ookinete invasion pathways, subtilisin-like protease 2 (SUB2) is thought to be a candidate to interfere with the disease-causing forms of malaria asexual development, as well as development in the obligate mosquito host. In merozoites, SUB2 accumulates in the parasite micronemes and is secreted onto the merozoite surface upon schizont rupture. There, SUB2 interacts with an actin-dependent motor to behave as a sheddase, cleaving surface-bound MSP1 and AMA1 on the parasite membrane. As SUB2 moves to the posterior end of the merozoite during RBC invasion, these substrates are cleaved at a certain distance relative to the membrane with minimal sequence specificity, in contrast to other proteases. Little is known regarding SUB2 function during ookinete invasion, and limited evidence suggests that it is secreted by ookinetes during mosquito midgut invasion. In cells that have undergone ookinete invasion, SUB2 is found in protein aggregates in close association with the actin cytoskeleton and may function to disrupt the host cytoskeletal network to facilitate invasion. Further evidence to define SUB2's role in the sexual stages of parasite development have yet to be explored.

### SUMMARY OF THE INVENTION

In accordance with an embodiment, the present invention provides an immunogenic composition comprising one or more subtilisin-like protease 2 antigens from Plasmodium.

In accordance with another embodiment, the present invention provides an immunogenic composition comprising one or more subtilisin-like protease 2 antigens, wherein the subtilisin-like protease 2 antigens are selected from P. berghei, P. falciparum, P. vivax, P. knowlesi, and P. yoelli.

In accordance with a further embodiment, the present invention provides an immunogenic composition comprising one or more subtilisin-like protease 2 antigens, wherein the subtilisin-like protease antigen has an amino acid sequence selected from the group consisting of SEQ ID NOS: 1-12.

In accordance with an embodiment, the present invention provides a vector comprising one or more subtilisin-like protease 2 antigens from Plasmodium.

In accordance with another embodiment, the present invention provides a cell expressing the vector described herein.

In accordance with a further embodiment, the present invention provides a method of blocking transmission of a Plasmodium infection in a subject comprising administering to the subject an immunogenic composition comprising one or more subtilisin-like protease 2 antigens from Plasmodium, thereby blocking transmission of Plasmodium infection in the subject.

In accordance with an embodiment, the present invention provides a method of immunizing a subject against Plasmodium infection comprising administering to a subject an immunogenic composition comprising one or more composition comprising one or more subtilisin-like protease 2 antigens from Plasmodium, thereby blocking transmission of Plasmodium infection in the subject.

In accordance with a further embodiment, the present invention provides a method for treating or preventing malaria in a subject comprising administering to a subject an immunogenic composition comprising one or more composition comprising one or more subtilisin-like protease 2 antigens from Plasmodium, thereby blocking transmission of Plasmodium infection in the subject.

### BRIEF DESCRIPTION OF THE DRAWINGS

FIG. 1 shows the PbSUB2 homology models used to identify peptide targets for immunization. (1A) Illustration (left) or surface representation (right) homology model of the PbSUB2 catalytic domain (residues L672-L971) visualized with PyMOL software. Loop regions corresponding to Peptide #1 highlighted in purple and Peptide #2 in green were used for immunization experiments. Catalytic residues Asp705, His748 and S911 in the active site pocket are shown as orange, cyan and red spheres respectively. (1B) Lateral view of Peptide #1 (purple) and Peptide #2 (green) in the PbSUB2 surface representation model reveals that each peptide corresponds to solvent exposed areas of PbSUB2 (left). Sequence alignments of PbSUB2 Peptide #1 (top) and Peptide #2 (bottom) sequences with corresponding regions of P. falciparum, P. vivax, P. knowlesi, and P. yoelii SUB2(right). The amino acid position of the first and last residues of each peptide sequence with respect to full length PbSUB2 are shown at the top left and right corner of each alignment (K 723 and D 736 for Peptide #1; R 946 and I 959 for Peptide #2). Conserved residues are highlighted with a red background and regions of similarity are marked with red letters against white background.

FIG. 2 shows production of recombinant SUB2 and recognition using Sub2 immune sera. (2A) Domains of endogenous PbSUB2 (top): signal peptide (residues 1-20), prodomain (residues 21-626), catalytic domain (residues 627-1088) with catalytic residues Asp (orange), His (cyan) and Ser (red), transmembrane domain (residues 1089-1111) and cytoplasmic tail (residues 1112-1230). Representation of recombinant PbSUB2 (middle) containing a minimal inhibitory domain and the full catalytic domain (active site residues not pictured). Below, PbSUB2 Peptides #1 (purple) and #2 (green) are aligned to endogenous PbSUB2 and rPbSUB2 with peptide sequences. (2B) Recombinant proteins maltose binding protein (MBP), PbSUB2 or PfSUB2 MBP-fusion proteins were separated on polyacrylamide gels and stained with Coomassie, or transferred and visualized by Western Blot with specific MBP, SUB2, or KLH antibodies. Arrows denote full length PbSUB2 and PfSUB2 recombinant products. Approximate sizes in kilodaltons (kDa) are displayed on the left.

FIG. 3 depicts P. berghei development is attenuated in SUB2-immunized mice. The parasitemia of KLH- or SUB2-immunized mice was determined over the period of ten days after infection with 2×10² P. berghei parasites. Results are shown for KLH- and SUB2-immunized mice using the IFA (n=6) (3A), or CFA (n=3) (3B) immunization protocols. Each point represents the mean parasitemia with error bars displaying standard errors of the mean. The scatter plot displays the parasitemia at Day 10, with each point representing the parasitemia of individual KLH- or SUB2-immunized mice for each immunization protocol. The red bar represents the median of each experiment.

FIG. 4 shows SUB2-immunization promotes multiple invasion of red blood cells. Representative images of single, double, or multiple invasion (3+) events in P. berghei infected red blood cells (top). The percentage of infected RBCs displaying the single, double, or multiple invasion phenotypes in the KLH- or SUB2-immunized mice (CFA protocol) is displayed below each image. The mean and standard error are displayed for each experimental treatment, with asterisks denoting significance.\

FIG. 5 depicts SUB2-immunized mice have increased survival upon malaria parasite challenge. Survival curves of KLH- and SUB2-immunized mice using the IFA (5A) or CFA immunization protocols (5B) over the course of forty days following P. berghei challenge. Black or grey dashed lines denote the mean survival time for KLH- or SUB2-immunized mice respectively.

FIG. 6 shows that passive immunization with SUB2 immune sera does not influence parasite growth in the mosquito. Oocyst numbers were measured to determine the effects of passive immunization to control KLH- or SUB2-immune sera. P. berghei-infected mice were fed to mosquitoes and oocyst numbers were determined for each experimental group before passive immunization (pre-KLH or pre-SUB2), or following passive immunization (KLH or SUB2). Oocyst numbers from two independent experiments were pooled and analyzed by Kruskal-Wallis with a Dunn's Multiple Comparison test to determine significance. The total number (n) of mosquito midguts examined is displayed under each experimental group. The bar denotes the median of each experiment. No significant (ns) differences were identified for either experimental group following passive immunization.

### DETAILED DESCRIPTION OF THE INVENTION

Plasmodium species utilize many different proteases during their complex life cycle in the human and mosquito hosts, and serve as optimal targets to interfere with malaria transmission.

Using a rodent model, the present inventors address the potential of targeting SUB2 by immunizing mice against specific SUB2 derived peptides. When compared to control KLH-immunized mice, SUB2-immunization resulted in a slight delay in pre-patency, decreased parasitemia when monitored over a ten day period, and increased survival following infection. Similar results were obtained independent of the method of immunization, suggesting that the effects of immunization are primarily that of the SUB2 antigens and not from non-specific effects mediated by the CFA. Together, these data show that SUB2-immunization greatly impairs parasite growth, likely by interfering with the efficacy of merozoite invasion.

In support of this idea, the inventors detected an increase in the number of multiply invaded RBCs following SUB2-immunization, suggesting that merozoite invasion is significantly altered. Similar effects have been seen in other studies using antibodies targeting merozoite proteins, where it was proposed that multiple invasions are the result of merozoite agglutination. According to our hypothesis, the invasion of some merozoites may be completely blocked, while incomplete inhibition may result in multiple parasites that have been cross-linked by SUB2 antibodies, undergo invasion together as a complex or dissociate once the RBC surface has been recognized. Due to the short time frame in which merozoites undergo release and invasion into new RBCs, the concentration and rate of antibody binding may be critical factors in invasion inhibition.

Very little information exists regarding the viability of infected RBCs that have undergone multiple invasions. Without being held to any particular theory, it has been hypothesized that nutritional and structural limitations following multiple invasion may reduce the production of viable merozoites, thus raising the possibility that these infected RBCS may be a “dead-end” for the parasite. As a result, the higher incidence of multiple invasions may have a significant contribution to the decreased parasitemia and increased survival in the SUB2-immunized mice within the compositions and methods of the present invention.

In accordance with an embodiment, the present invention provides an immunogenic composition comprising one or more subtilisin-like protease 2 antigens from Plasmodium.

The subtilisin-like protease 2 is expressed in Plasmodium and is a 147 kD polypeptide. PfSUB2 is a secreted type 1 integral membrane protein. Importantly, PfSUB2 contains the four consensus sequences known to form the active site of subtilisin-like serine proteases of the superfamily S8.

In accordance with another embodiment, the present invention provides an immunogenic composition comprising one or more subtilisin-like protease 2 antigens, wherein the subtilisin-like protease 2 antigens are selected from P. berghei, P. falciparum, P. vivax, P. knowlesi, and P. yoelli.

As used herein, the term “subtilisin-like protease 2 antigens” means a portion or fragment of subtilisin-like protease 2 protein that is capable of binding an antibody or portion or fragment thereof.

In one or more embodiments, the subtilisin-like protease 2 antigens comprise SEQ ID NOS: 1-12, as disclosed herein.

The subtilisin-like protease 2 antigens can also include functional fragments, functional homologs and fusion polypeptides comprising an amino acid sequence of SEQ ID NOS: 1-12, as disclosed herein.

The term, “amino acid” includes the residues of the natural α-amino acids (e.g., Ala, Arg, Asn, Asp, Cys, Glu, Gln, Gly, His, Lys, Ile, Leu, Met, Phe, Pro, Ser, Thr, Trp, Tyr, and Val) in D or L form, as well as β-amino acids, synthetic and non-natural amino acids. Many types of amino acid residues are useful in the polypeptides and the invention is not limited to natural, genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC.

Reference herein to “derivatives” includes parts, fragments and portions of the inventive subtilisin-like protease 2 antigens. A derivative also includes a single or multiple amino acid substitution, deletion and/or addition. Homologues include functionally, structurally or sterochemically similar peptides from the same species of parasite or from within the same genus or family of parasite. All such homologues are contemplated by the present invention.

Examples of incorporating non-natural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A partial list of known non-natural amino acid contemplated herein is shown in Table 1.

TABLE 1
Non-natural Amino Acids

| Non-conventional | |
|---|---|
| amino acid | Code |
| α-aminobutyric acid | Abu |
| α-amino-a-methylbutyrate | Mgabu |
| aminocyclopropane- | Cpro |
| carboxylate | |
| aminoisobutyric acid | Aib |
| aminonorbomyl- | Norb |
| carboxylate | |
| cyclohexylalanine | |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| Chexa L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Analogs of the subject peptides contemplated herein include modifications to side chains, incorporation of non-natural amino acids and/or their derivatives during peptide synthesis and the use of crosslinkers and other methods which impose conformational constraints on the peptide molecule or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

The present invention can also include non-naturally occurring peptide analogs of the subtilisin-like protease 2 antigens disclosed herein. As used herein, the term “non-naturally occurring” means that the amino acid sequence of the antigens, homologs, or functional portions thereof, are not found in nature.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference. In some embodiments the portion retains at least 50%, 75%, or 80%, or more preferably 90%, 95%, or even 99% of the biological activity of the reference protein or nucleic acid described herein.

The term “host cell” is meant to refer to a cell into which a foreign gene is introduced. The host cell can be prokaryotic or eukaryotic. In preferred embodiments, the host cell is E.coli or an E.coli derivative.

By “immunogenic composition” is meant to refer to one or more Plasmodium subtilisin-like protease 2 antigens that are capable of eliciting protection against malaria, whether partial or complete. An immunogenic composition may also be useful for treatment of an infected individual.

The terms “isolated,” “purified,” or “biologically pure,” refer to material that is free to varying degrees from components which normally accompany it as found in its native state. Various levels of purity may be applied as needed according to this invention in the different methodologies set forth herein; the customary purity standards known in the art may be used if no standard is otherwise specified.

By “isolated nucleic acid molecule” is meant a nucleic acid (e.g., a DNA, RNA, or analog thereof) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “nucleic acid” is meant an oligomer or polymer of ribonucleic acid or deoxyribonucleic acid, or analog thereof This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

The term “complimentary nucleic acid sequences” refer to contiguous DNA or RNA sequences which have compatible nucleotides (e.g., A/T, G/C) in corresponding positions, such that base pairing between the sequences occurs. For example, the sense and anti-sense strands of a double-stranded DNA helix are known in the art to be complimentary.

By “protein” is meant any chain of amino acids, or analogs thereof, regardless of length or post-translational modification.

By “reference” is meant a standard or control condition.

By “specifically binds” is meant a molecule (e.g., peptide, polynucleotide) that recognizes and binds a protein or nucleic acid molecule of the invention, but which does not substantially recognize and bind other molecules in a sample, for example, a biological sample, which naturally includes a protein of the invention.

As used herein, the terms “treat,” “treating,” “treatment,” and the like refer to reducing or ameliorating a disorder and/or symptoms associated therewith, for example malaria. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition, for example malaria.

The present invention features immunogenic compositions comprising one or more one or more subtilisin-like protease 2 antigens, wherein the subtilisin-like protease 2 antigens are selected from P. berghei, P. falciparum, P. vivax, P. knowlesi, and P. yoelli.

It has been discovered that a nucleotide sequence capable of enhanced expression in host cells can be obtained by harmonizing the frequency of codon usage in the foreign gene at each codon in the coding sequence to that used by the host cell. In certain embodiments, the invention features a nucleic acid sequence encoding a polypeptide to enhance expression and accumulation of the polypeptide in the host cell. Accordingly, the present invention provides novel nucleic acid sequences, encoding a polypeptide or protein that is foreign to a host cell, and that is expressed at greater levels and with greater biological activity than in the host cell as compared to the wild-type sequence if expressed in the same host cell.

Certain examples of codon harmonization have been described, for example, in US Application Nos. 20060088547 and 20080076161 and Angov et al. (PLOS, 2008. Volume 3, issue 5), which are incorporated by reference in their entireties herein. The methods of the present invention, while directed to codon harmonization of subtilisin-like protease 2 antigens, are not limited as such, and are applicable to any coding sequence encoding a protein foreign to a host cell in which the protein is expressed.

Accordingly, in certain embodiments the invention features a method for preparing a codon harmonized subtilisin-like protease 2 antigen sequence encoded by Plasmodium from a subtilisin-like protease 2 gene comprising determining the frequency of codon usage of the subtilisin-like protease 2 gene coding sequence, and substituting codons in the coding sequence with codons of similar frequency from a host cell which code for the same subtilisin-like protease 2 antigen, thereby preparing a codon harmonized subtilisin-like protease 2 antigen sequence.

For example, the frequency of occurrence of each codon in the Plasmodium subtilisin-like protease 2gene of interest can be calculated and replaced with an E. coli codon with a similar frequency for the same amino acid.

An existing DNA sequence can be used as the starting material and modified by standard mutagenesis methods that are known to those skilled in the art or a synthetic DNA sequence having the desired codons can be produced by known oligonucleotide synthesis, PCR amplification, and DNA ligation methods.

The compositions of the invention are designed for expression in a host. In preferred embodiments, a host is E.coli or an E.coli derivative. The DNA encoding the desired recombinant protein can be introduced into a host cell in any suitable form including, the fragment alone, a linearized plasmid, a circular plasmid, a plasmid capable of replication, an episome, RNA, etc. Preferably, the gene is contained in a plasmid. In a particularly preferred embodiment, the plasmid is an expression vector. Individual expression vectors capable of expressing the genetic material can be produced using standard recombinant techniques. Please see e.g., Maniatis et al., 1985 Molecular Cloning: A Laboratory Manual or DNA Cloning, Vol. I and II (D. N. Glover, ed., 1985) for general cloning methods

In accordance with another embodiment, the present invention provides a cell expressing the vector described herein.

In accordance with a further embodiment, the present invention provides an immunogenic composition comprising one or more one or more subtilisin-like protease 2 antigens, wherein the subtilisin-like protease 2 antigens are selected from P. berghei, P. falciparum, P. vivax, P. knowlesi, and P. yoelli in a conjugate vaccine composition. Conjugate vaccines typically consist of polysaccharides, generally from the surface coat of bacteria or other target organism, linked to protein carriers. The combination of the polysaccharide and protein carrier induces an immune response against the target organism displaying the polysaccharide contained within the vaccine on their surface, thus preventing disease.

Thus, in an embodiment, the present invention provides at least one or more subtilisin-like protease 2 antigens covalently linked to another known antigen, such as, for example, Hepatitis B surface antigen. Such conjugate vaccines are known by those of ordinary skill in the art and methods for making them can be found in WO1993/010152, which describes the RTS,S/AS01 vaccine for malaria, which is in clinical trials.

The immunogenic compositions of the present invention can be administered to a subject by different routes such as subcutaneous, intradermal, intramuscular, intravenous and transdermal delivery. Suitable dosing regimens are preferably determined taking into account factors well known in the art including age, weight, sex and medical condition of the subject; the route of administration; the desired effect; and the particular composition. The course of the immunization may be followed by assays for activated T cells produced, skin-test reactivity, antibody formation or other indicators of an immune response to a malarial strain.

Dosage form, such as injectable preparations (solutions, suspensions, emulsions, solids to be dissolved when used, etc.), tablets, capsules, granules, powders, liquids, liposome inclusions, ointments, gels, external powders, sprays, inhalation powders, eye drops, eye ointments, and the like, can be used appropriately depending on the administration method. Pharmaceutical formulations are generally known in the art and are described, for example, in Chapter 25.2 of Comprehensive Medicinal Chemistry, Volume 5, Editor Hansen et al, Pergamon Press 1990.

Pharmaceutically acceptable carriers which can be used in the present invention include, but are not limited to, an excipient, a stabilizer, a binder, a lubricant, a colorant, a disintegrant, a buffer, an isotonic agent, a preservative, an anesthetic, and the like which are commonly used in a medical field Immunogenic compositions are administered in immunologically effective amounts. An immunologically effective amount is one that stimulates the immune system of the subject to establish a level of immunological response sufficient to reduce parasite density and disease burden caused by infection with the pathogen, and/or sufficient to block the transmission of the pathogen in a subject. A dose of the immunogenic composition may, in certain preferred embodiments, consist of the range of 1 μg to 1.0 mg total protein. In certain preferred embodiments, the composition is administered in a concentration between 1-100 μg. However, one may prefer to adjust dosage based on the amount of antigen delivered. In either case these ranges are guidelines. More precise dosages should be determined by assessing the immunogenicity of the composition so that an immunologically effective dose is delivered. The immunogenic composition can be used in multi-dose formats.

The timing of doses depends upon factors well known in the art. After the initial administration one or more booster doses may subsequently be administered to maintain antibody titers, e.g., the compositions of the present invention can be administered one time or serially over the course of a period of days, weeks, months and or years. An example of a dosing regime would be day 1 an additional booster doses at distant times as needed. The booster doses may be administered at 2, 3, 4, 5, 6, 7, 8, 9, 10, 11, 12, 13, 14, 15, 16, 17, 18, 19, 20 or more weeks after the primary immunization. In preferred embodiments, the booster doses are administered at 4 weeks. In other preferred embodiments, the booster doses are administered at 12 weeks.

As such, in accordance with an embodiment, the present invention provides a method of immunizing a subject against Plasmodium infection comprising administering to a subject an immunogenic composition comprising one or more composition comprising one or more one or more subtilisin-like protease 2 antigens from Plasmodium, thereby blocking transmission of Plasmodium infection in the subject.

In accordance with an embodiment, the present invention provides the use of the immunogenic composition comprising one or more one or more subtilisin-like protease 2 antigens from Plasmodium, to block transmission of Plasmodium infection in the subject comprising administering to the subject the immunogenic composition.

As used herein the subject that would benefit from the immunogenic compositions described herein include any host that can benefit from protection against malarial infection. Preferably, a subject can respond to inoculation with the immunogenic compositions of the present invention by generating an immune response. The immune response can be completely or partially protective against symptoms caused by infection with a pathogen such as Plasmodium falciparum, or can block transmission of the pathogen by Anopheles mosquitoes. In a preferred embodiment, the subject is a human. In another embodiment, the subject is a non-human primate.

The immunogenic compositions of the present invention can be used to immunize mammals including humans against infection and/or transmission of malaria parasite, or to treat humans post-infection, or to boost a pathogen-neutralizing immune response in a human afflicted with infection of malaria parasite.

In accordance with another embodiment, the present invention provides the use of the immunogenic composition described herein for treating or preventing malaria in a subject comprising administering to a subject the immunogenic compositions.

The immunogenic compositions of the present invention can be formulated according to methods known and used in the art. Guidelines for pharmaceutical administration in general are provided in, for example, Modern Vaccinology, Ed. Kurstak, Plenum Med. Co. 1994; Remington's Pharmaceutical Sciences 18th Edition, Ed. Gennaro, Mack Publishing, 1990; and Modern Pharmaceutics 2nd Edition, Eds. Banker and Rhodes, Marcel Dekker, Inc., 1990 Immunogenic compositions of the present invention can be prepared as various salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as histidine, arginine and lysine.

Adjuvants are almost always required to enhance and/or properly direct the immune response to a given antigen. An ideal adjuvant should be safe, stable with long shelf life, biodegradable, inexpensive and promote an appropriate immune response while itself being immunologically inert. Adjuvants affect processes including antigen presentation, antigen uptake and selective targeting of antigens thus critically determining the magnitude and type of the immune responses. While the mechanisms by which different adjuvants result in different outcomes remain a “black box”, studies strive for developing a vaccine that can provide maximum efficacy with ease of delivery in as fewer doses as possible. It must be kept in mind that an adjuvant is not the active component in a vaccine and immunization; outcomes can vary greatly from one adjuvant to another when used in combination with the same vaccine antigen. Any given adjuvant—vaccine combination has to be evaluated on a case-by-case basis for safety, reactogenicity and efficacy in pre-clinical trials. Ultimately, safety considerations outweigh any anticipated benefit and need to be evaluated for the development of a plan leading to human clinical trial.

In certain preferred embodiments, the immunogenic compositions are formulated with an aluminum adjuvant. Aluminum based adjuvants are commonly used in the art and include aluminum phosphate, aluminum hydroxide, aluminum hydroxy-phosphate, and amorphous aluminum hydroxyphosphate sulfate. Trade names of aluminum adjuvants in common use include ADJUPHOS, ALHYD ROGEL, (both from Superfos Biosector a/s, DK-2950 Vedbaek, Denmark).

Non-aluminum adjuvants can also be used. Non-aluminum adjuvants include, but are not limited to, QS21, Lipid-A, Iscomatrix, and derivatives or variants thereof, Freund's complete or incomplete adjuvant, neutral liposomes, liposomes containing vaccine and cytokines or chemokines.

Emulsions of Montenide ISA 51 (a mineral oil adjuvant) and ISA 720 (oil-based non-mineral oil) have been used in human clinical trials. A review of clinical trials (25 trials representing more than 4000 patients and 40,000 injections for Montanide ISA 51 and various trials representing 500 patients and 1500 injections for Montanide ISA 720) has revealed their general safety and strong adjuvant effect with mild to moderate local reactions.

In certain preferred embodiments of the invention, the method of the invention further comprises administering an adjuvant. In certain examples, the adjuvant is selected a water-in-oil emulsion. In other examples, the adjuvant is Aluminum hydroxide. However, any adjuvant that is suitable for administration with the immunogenic composition in the methods of the present invention can be suitably used.

In accordance with a further embodiment, the present invention provides a method of blocking transmission of a Plasmodium infection in a subject comprising administering to the subject an immunogenic composition comprising one or more subtilisin-like protease 2 antigens from Plasmodium, thereby blocking transmission of Plasmodium infection in the subject.

As used herein, the term “blocking transmission” means that the antibodies to the subtilisin-like protease 2 antigens” interfere with the disease-causing forms of malaria asexual development, as well as development in the obligate mosquito host.

In accordance with a further embodiment, the present invention provides a method for treating or preventing malaria in a subject comprising administering to a subject an immunogenic composition comprising one or more composition comprising one or more subtilisin-like protease 2 antigens from Plasmodium, thereby blocking transmission of Plasmodium infection in the subject.

As used herein, the other compositions which can be used in conjunction with the compositions and methods disclosed herein include, for example, quinine, quinidine, chloroquine, amodiaquine, pyrimethamine, proguanil, sulfonamides such as sulfadoxine and sulfamethoxypyridazine, mefloquine, atovaquone, primaquine, artemisinin and its derivatives artemether, artesunate, and dihydroartemisinin, halofantrine, doxycycline, and clindamycin.

### EXAMPLES

SUB2 homology modeling and visualization. Homology model of PbSUB2 (PlasmoDB code: PBANKA_091170, Gene ID: 3423789) was generated using the I-TASSER Protein Structure and Function Prediction Server using default settings (BMC Bioinformatics 9:40 (2008)). From all the models predicted by the server, the one with the highest confidence score was used in our study. Models were visualized using PyMol (The PyMoL Molecular Graphics System, Version 1.6.0.0 Schrödinger, LLC).

Mice. Female Swiss Webster mice (˜21-24 g) were purchased from Harlan and maintained in accordance with the recommendations of the Guide for the Care and Use of Laboratory Animals of the National Institutes of Health. All animal procedures were approved by the Institutional Animal Care and Use Committee of the Johns Hopkins University (protocol number MO09H58).

SUB2 immunization. Synthetic SUB2 peptides conjugated to keyhole limpet hemocyanin (KLH) through the cysteine at the N-(Sub2 Peptide #2-CRTSIKIVSKDKKTI) (SEQ ID NO: 12) or C-terminus (Sub2 Peptide #1-KYSDRYEMTDELFDC) (SEQ ID NO: 11) via a —SH bond were produced by GenScript Corporation (Piscataway, N.J.).

Female Swiss Webster mice (˜21-24 g) were primed with a 50:50 mixture (50 μg/mouse) of both SUB2 peptides in phosphate buffered saline (PBS) or 5 μg of a control KLH carrier in PBS with either complete Freund's adjuvant (CFA) or incomplete Freund's adjuvant (IFA) in a 1:1 emulsion and immunized by Intra-peritoneal injection (i.p.). Mice were boosted four times in two week intervals with 50 μg/mouse of peptide in a 1:1 emulsion with IFA via i.p. injection. Serum was collected from each individual mouse prior to priming, as well as the third and fourth boosting immunizations to monitor antibody titers. Two weeks after the final boosting immunization, animals were used for subsequent challenge experiments with P. berghei parasites.

P. berghei and P. falciparum RNA isolation and cDNA production. P. berghei ANKA 2.34 total RNA was prepared from blood of an infected Swiss Webster mouse (˜10% parasitemia) obtained via cardiac puncture and isolated using TRIzol Reagent (Invitrogen) according to the manufacturer's specifications. Two μg of total RNA was used as a template for the production of cDNA using SuperScriptIII (Invitrogen, Carlsbad, Calif.).

Approximately 1 μg of total RNA from asynchronized P. falciparum 3D7 parasites was isolated using TRI Reagent (Molecular Research Center, Inc. Cincinnati, Ohio) and treated with DNase I (New England Biolabs, Ipswich, Mass.) according to the manufacturer's protocol. Synthesis of complementary DNA was performed with the SuperScript First-Strand Synthesis System for RT-PCR (Invitrogen).

Plasmodium SUB2 cloning. P. berghei SUB2 N476-N1185 (PlasmoDB code: PBANKA_091170, Gene ID: 3423789) and P. falciparum SUB2 N528-S1135 (PlasmoDB code: PF3D7_1136900, Gene ID: 810927) coding sequences were amplified using cDNA obtained from P. berghei ANKA 2.34 or P. falciparum 3D7 strains using the respective primers PbSUB2_Fwd: 5′ CTCCATGGCGAATAATTCAAATGCATTTTTGAGTGTAGAC 3′, (SEQ ID NO: 13) PbSUB2_Rev: 5′ ACGGATCCGTTATCATGCTCATATAAATTATATAAAGC 3′, (SEQ ID NO: 14) PfSUB2_Fwd: 5′ ATCCATGGCGAATAATAAAAAAATTTTGTTAAATGTTGAT 3′ (SEQ ID NO: 15) and PfSUB2_Rev: 5′ ACGGATCCACTATCATATTCATACAAATTATATAAGGC 3′ (SEQ ID NO: 16). PCR products were amplified using Phusion® High-Fidelity DNA polymerase (New England Biolabs) with an annealing temperature gradient of 52° C.-70° C. for 30 seconds, followed by extension at 72° C. for 2 minutes.

SUB2 PCR products were inserted in frame using NcoI and BamHI restriction sites into a modified pRSF-1b vector (Novagen) for expression as an Maltose Binding Protein (MBP)-fusion protein with a C-terminal 6×His tag for purification and detection purposes as previously described (J. Mol. Recognit. 26:496-500 (2013)). Positive clones were screened using colony PCR with primers described above and insertion sequences were confirmed by sequencing.

Recombinant protein expression and purification. MBP-SUB2 fusion constructs were transformed into Rosetta 2 (DE3) competent E. coli (Novagen) for protein expression. Cells were grown in the presence of 1.5% glucose and 50 μg/ml kanamycin in 500 ml 1× Terrific Broth media until OD₆₀₀ of ˜3.0 and induced with a final concentration of 0.5 mM IPTG. Recombinant proteins were expressed overnight at 20° C. under vigorous shaking at 250 rpm.

Bacteria were harvested by centrifugation at 2,500 RPM for 30 minutes at 4° C. Bacterial pellets were re-suspended in lysis buffer (25 mM Tris pH 9.0, 100 mM NaCl) and lysis was performed using an Emulsiflex C5 cells disruptor (Avestin Inc., Ottawa, Canada) at 100 MPa. Whole cell lysates were fractionated by centrifugation at 17,000 rpm for 1 hour at 4° C. and the supernatant was applied to an open BioRad gravity column containing 1 ml of Amylose resin (New England Biolabs) for affinity capture of the MBP taged fusion protein. Bound protein was washed with lysis buffer and eluted in the presence of 20 mM maltose. Elution samples from the Amylose resin purification steps were applied to an affinity column containing Cobalt-TALON resin (Clontech, Mountain View, Calif.) for secondary purification with the 6×His tag. Bound protein was washed with lysis buffer and eluted with 200 mM imidazole. Elution samples were concentrated using Nanosep Centrifugal Devices (Sigma) with a 10 kDa cutoff

Western blots. Approximately 1.7 μg of recombinant PbSUB2 and PfSUB2, and ˜3 μg MBP (fusion protein only) were separated on a 12% SDS-PAGE gel. Following electrophoresis, the gel was washed in diH₂O for 10 minutes and equilibrated in 1× transfer buffer (25 mM Tris, 192 mM Glycine, 20% methanol, 0.0375% SDS). Proteins were transferred to a PVDF membrane on a Semi-dry transfer cell for 2 hours under constant voltage (25V). After transfer, the membrane was blocked with 5% milk in 1× TBST for 30 minutes (250 rpm at 37° C.) and washed three times with 1× TBST. Membranes were incubated overnight at 4° C. with serum from SUB2- or KLH-immunized mice at a 1:500 dilution in 1× TBST or with a mouse anti-Maltose Binding Protein antibody (Upstate—Millipore, #05-912) at a 1:10,000 dilution in 1× TBST. After three washes with 1× TBST, membranes were incubated with an alkaline phosphatase-conjugated goat anti-mouse antibody (1:5,000 dilution in 1× TBST). Detection was carried out using NBT/BCIP alkaline phosphatase substrates (Promega, Madison, Wis.).

Plasmodium challenge in SUB2 immunized mice. Following immunization with either the CFA or IFA protocols described above, SUB2 or control KLH mice were infected with ˜2×10² P. berghei mCherry (Biotechnol. J. 4:895-902 (2009)) asexual parasites via intra venous (IV) injection as previously performed (PLoS Pathog 5:e1000302 (2009)). To monitor parasite growth, thin smears of tail blood were stained with Giemsa and examined under a microscope to determine parasitemia (% of infected erythrocytes) every day for ten days. Results were combined for KLH- and SUB2-immunized mice using either the IFA or CFA immunization protocols and significance was determined using linear regression analysis. Statistical comparisons of the parasitaemia at day 10 of infected mice were performed using Mann-Whitney analysis.

To determine the effects of immunization on mouse survival following the above Plasmodium challenge, the survival of immunized mice was monitored for 40 days following the initial infection. Statistical differences in the survival curves were determined using a Log-rank (Mantel-Cox) test.

Multiple Invasion Analysis. Ten days after infection with P. berghei, Giemsa-stained thin smears from SUB2 or KLH CFA immunized mice were analyzed under a microscope. Independent of parasitemia, at least 200 infected RBCs were examined per mouse to determine the number of infected RBCs that contain one or more parasites. The percentage of each invasion phenotype was calculated as the number of invasion events, divided by the total number of infected RBCs (iRBCs). Significance was determined using Mann-Whitney test.

Passive immunization experiments. Swiss Webster mice infected with the mCherry strain of P. berghei were examined for similar levels of exflagellation three days after inoculation as previously described (Proc. Natl. Acad. Sci. U.S.A. 104:13461-6 (2007)). Mice with matching infections were anesthetized and used for blood feeding control (pre-KLH) or treatment (pre-SUB2) groups of An. gambiae mosquitoes for 15 minutes. The anesthetized mice were then taken off the cage and passively immunized (i.v.) with KLH or SUB2 immune sera (final concentration of 2 mg/ml) and allowed to recover for 15 minutes. The passively immunized mice were then fed to sibling groups of An. gambiae mosquitoes for an additional 15 minutes to measure any effects on parasite development in the mosquito.

Following feeding, mosquitoes were incubated at 19° C. to promote P. berghei development. Mosquito midguts were dissected 7 days post-blood meal (PBM), and oocysts numbers were counted using a compound fluorescence microscope. Oocyst numbers from two independent experiments were pooled and analysed by Kruskal-Wallis with a Dunn's Multiple Comparison test to determine significance.

### Example 1

Structural modeling of P. berghei SUB2 catalytic domain.

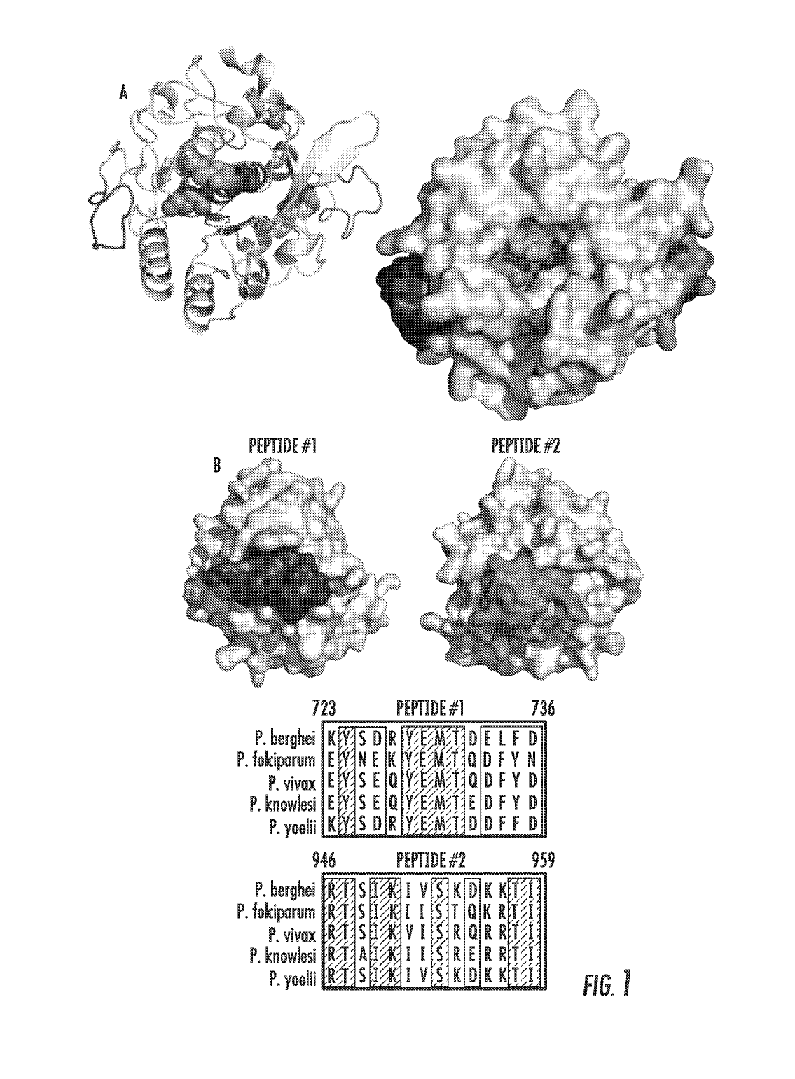

A structure model was predicted for the catalytic domain of PbSUB2 by the I-TASSER server and contains a secondary structure topology characteristic of subtilisin-like serine proteases (FIG. 1A). The amino acid residues that comprise the catalytic triad Asp 705, His 748 and Ser 911 required for catalysis are positioned at the active site of the model (FIG. 1A). Comparing our predicted model using the EBI SSM webserver, the closest structural homolog in the Protein Data Bank (PDB) is the subtilase, thermitase (PDB 1twc:E) from Thermoactinomyces vulgaris. With an overall root mean square deviation (R.M.S.D) of 1.4 Å for 247 amino acid residues as determined with PDBeFold (Acta Crystallogr. D. Biol. Crystallogr. 60:2256-68), our predicted structural model for PbSUB2 therefore has a high confidence level, resembling the overall known fold of other subtilases.

### Example 2

Design of P. berghei SUB2 peptides.

Using proprietary software (GenScript), highly antigenic peptides corresponding to the PbSUB2 catalytic domains were identified (Table 2). To test these candidate 14 amino acid peptides, the corresponding regions were mapped on a PbSUB2 catalytic domain homology model. Two representative peptides mapping to opposite flexible solvent exposed regions of PbSUB2 were selected to increase the likelihood that antibodies generated against these peptides would interact with the protease on the surface of merozoites or ookinetes during invasion (FIG. 1A). Peptide #1 and #2 target (SEQ ID NOS: 11-12) unique solvent accessible regions of the catalytic domain of PbSUB2 (FIG. 1B, left).

TABLE 2
Peptide Antigens
Peptide 1

| | 723 | | | | | | | | | | | | | 736 |
|---|---|---|---|---|---|---|---|---|---|---|---|---|---|---|
| P. | K | Y | S | D | R | Y | E | M | T | D | E | L | F | D (SEQ ID NO: 1) |
| berghei | | | | | | | | | | | | | | |
| P. | E | Y | N | E | K | Y | E | M | T | Q | D | F | Y | N (SEQ ID NO: 2) |
| falciparum | | | | | | | | | | | | | | |
| P. vivax | E | Y | S | E | Q | Y | E | M | T | Q | D | F | Y | D (SEQ ID NO: 3) |
| P. | E | Y | S | E | Q | Y | E | M | T | E | D | F | Y | D (SEQ ID NO: 4) |
| knowlesi | | | | | | | | | | | | | | |
| P. yoelii | K | Y | S | D | R | Y | E | M | T | D | D | F | F | D (SEQ ID NO: 5) |

Peptide 2

| | 946 | | | | | | | | | | | | | 959 |
|---|---|---|---|---|---|---|---|---|---|---|---|---|---|---|
| P. | R | T | S | I | K | I | V | S | K | D | K | K | T | I (SEQ ID NO: 6) |
| berghei | | | | | | | | | | | | | | |
| P. | R | T | S | I | K | I | I | S | T | K | K | R | T | I (SEQ ID NO: 7) |
| falciparum | | | | | | | | | | | | | | |
| P. vivax | R | T | S | I | K | V | I | S | R | R | R | R | T | I (SEQ ID NO: 8) |
| P. | R | T | A | I | K | I | I | S | R | R | R | R | T | I (SEQ ID NO: 9) |
| knowlesi | | | | | | | | | | | | | | |
| P. yoelii | R | T | S | I | K | I | V | S | K | D | K | K | T | I (SEQ ID NO: 10) |

The sequence of Peptide #1 is nearly identical (93%) to the corresponding region of P. yoelii Sub2 (FIG. 1B, right). The two sequences only differ by the amino acid at position Leu 734 in the P. berghei sequence and Phe 734 in P. yoelli, suggesting a high level of conservation between the rodent malaria species. Less conservation exists between Peptide #1 and the human malaria parasites (P. falciparum, P. vivax, and P. knowlesi), with only 64% identity (36% ID) to P. falciparum (FIG. 1B). However, the Peptide #2 sequence alignment reveals more conservation and sequence similarity across Plasmodium species. The P. berghei and P. falciparum SUB2 sequences show 85% identity (71% ID), while the rodent malaria parasites are completely conserved (FIG. 1B). Both peptide sequences map to regions of the PbSUB2 catalytic domain (FIG. 1A).

### Example 3

Mice immunized with SUB2 peptides recognize recombinant PbSUB2.

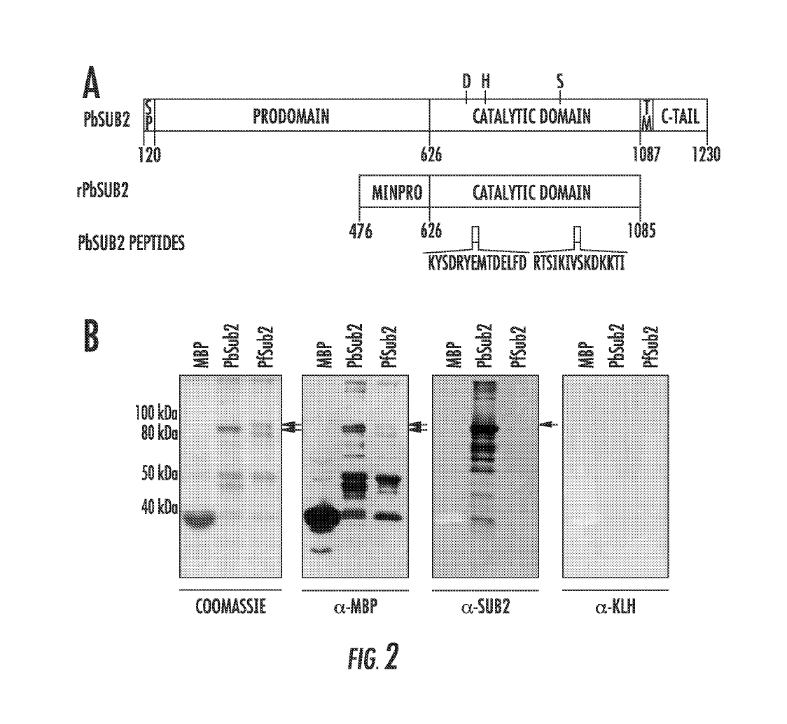

MBP-SUB2 expression constructs were expressed in Rosetta2 E. coli heterologous system as a single band for PbSUB2, or as two bands for PfSUB2, as approximate 110 kDa full-length protein products (FIG. 2B). Smaller protein products are likely the result of sample degradation during the purification process or translational truncation products that were observed for both SUB2 constructs (FIG. 2B). The truncation products can be explained by the occurrence of numerous rare-codons within the SUB2 gene, leading to premature termination during translation. Both full-length and truncated forms of SUB2 were detected using an MBP antibody, confirming the detection of the recombinant MBP-SUB2 fusion protein products (FIG. 2B). When incubated with immune sera from SUB2-immunized mice, recombinant PbSUB2 is detected in full length and degraded forms while only a faint band corresponding to full length recombinant PfSUB2 protein was detected (FIG. 2B). Importantly, mice immunized with KLH alone did not recognize either recombinant SUB2 protein (FIG. 2B).

These results confirm that antibodies were generated in mice immunized with PbSUB2 peptides that can sufficiently recognize recombinant PbSUB2 (FIG. 2B). Furthermore, immune sera raised against PbSUB2 peptides specifically targets PbSUB2 with minimal cross-reactivity to P. falciparum SUB2 (FIG. 2B), suggesting that the observed conservation in the peptide sequences is inadequate for cross-species protection. However, future immunization experiments are needed to determine the properties of the individual peptides and whether they are capable of cross-species immune recognition of different Plasmodium species.

### Example 4

SUB2-Immunization impairs asexual Plasmodium development.

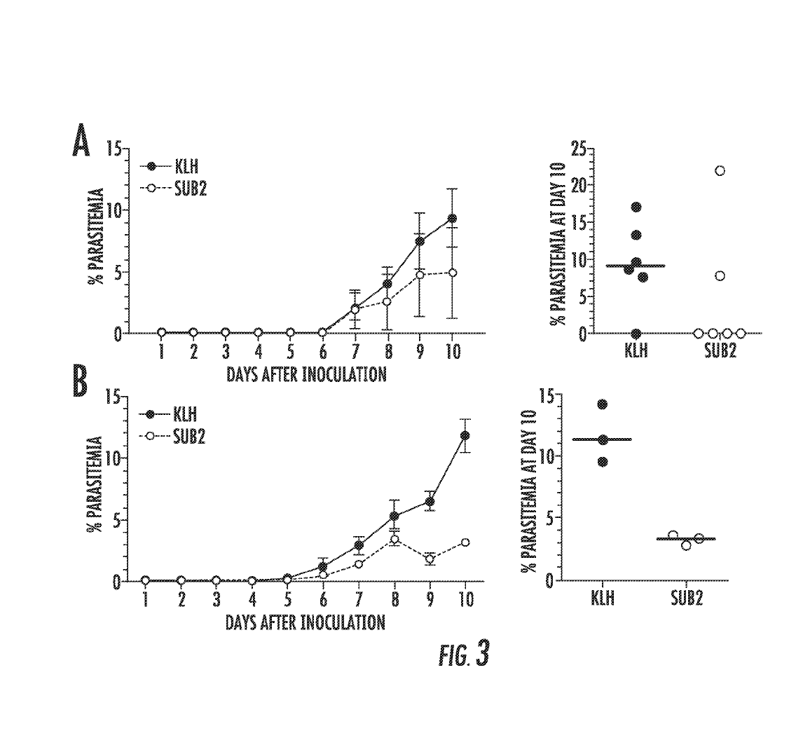

To monitor the effects of immunization on parasite development, KLH- and SUB2-immunized (IFA or CFA) mice were challenged with ˜2×10² P. berghei parasites by intravenous injection and the parasitaemia was monitored over the period of ten days. Blood stage infections were detected in 17 of 18 mice, and little variation was seen between mice immunized with the IFA or CFA immunization protocols (Table 3). As a result, both immunization experiments were pooled for analysis and are summarized (Table 3). Compared to control KLH-immunized mice, SUB2-immunized mice showed a slight, but not significant delay in the pre-patency of infection (Table 3). However, when the parasitaemia was monitored over the period of ten days, asexual growth was significantly reduced and in some mice completely attenuated following SUB2-immunization (FIG. 3A).

TABLE 3
Summary of Immunization Experiments

| | | | | | | | Mean |
|---|---|---|---|---|---|---|---|
| Experiment | Adjuvant | Antigen | #Mice | Infected | Pre-patency | Clearance* | survival^{§} |
| 1 | IFA | KLH | 6 | 5/6 | 6.2 | 0/5 | 27.3 |
| | | SUB2 | 6 | 6/6 | 6.7 | 4/6 | 34.6 |
| 2 | CFA | KLH | 3 | 3/3 | 6 | 0/3 | 31.3 |
| | | SUB2 | 3 | 3/3 | 6.7 | 0/3 | 40+ |
| Total | | KLH | 9 | 8/9 | 6.1 | 0/9 | 28.6 |
| | | SUB2 | 9 | 9/9 | 6.7 | 4/9 | 36.4 |

*Mice with detected parasitemia that had cleared the parasite infection (measured at day 10).
^{§}Average number of days mice survived following P. berghei challenge.

In mice following the IFA immunization protocol, parasite growth was reduced by 35, 36, and 48% from days 8-10 in the SUB2-immunized mice when compared to the KLH control (FIG. 3A). In addition, 4 out of the 6 SUB2-immunized mice had cleared the parasite infection by Day 10 (FIG. 3A, Table 3). Similar results were obtained in mice following the CFA immunization protocol, where parasite growth was reduced by 38, 71, and 73% from days 8-10 in the SUB2-immunized mice when compared to the KLH control (FIG. 3B). None of the KLH-immunized mice were able to clear the infection, the intensity of infection was reduced by ˜4 fold when compared to KLH control mice over the duration of the experiment (FIG. 3B).

Based upon these data and the important functional role of SUB2 in merozoite invasion, and without being held to any particular theory, we conclude that the reduced parasite growth in SUB2-immunized mice is presumably due to a decrease in the efficiency of merozoite invasion.

### Example 5

SUB2-immunization promotes abberant red blood cell invasion.

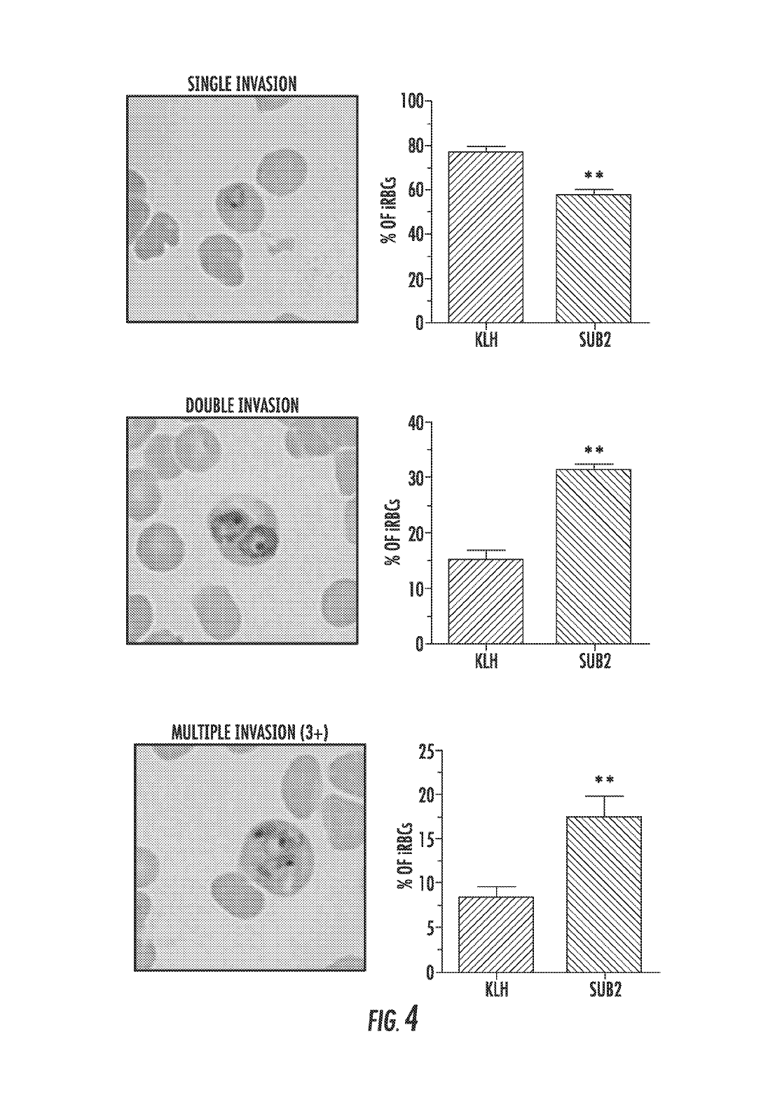

Based upon observations measuring the parasitemia of the immunized mice (FIG. 3), there appeared to be a noticeable increase in the number of infected RBCs with multiple parasites in SUB2-immunized mice. To quantify these presumed defects in invasion, the percentages of infected RBCs that had one, two, or multiple (3+) parasites were measured in the KLH- and SUB2-immunized mice following the CFA protocol. As previously observed, SUB2-immunized mice had a significant decrease in the number of infected RBCs that had undergone a single invasion event when compared to KLH-control mice (FIG. 4). In turn, corresponding increases in the number of double or multiple invasion events (three+) following SUB2 immunization were also detected (FIG. 4).

Based upon these data and the important functional role of SUB2 in RBC invasion, it is clear that SUB2-immunization interferes with merozoite invasion. Although it is not completely understood how SUB2-immunization might influence the production of these aberrant invasion events, previous studies using antibodies to merozoite surface proteins similarly report phenotypes promoting multiple invasion.

### Example 6

SUB2-Immunized mice have increased survival upon malaria parasite challenge.

Given that P. berghei asexual development is attenuated in SUB2-immunized mice (FIG. 3) and that this may be mediated in part by an increase in multiple invasion events (FIG. 4), we wanted to explore whether SUB2-immunization also results in increased survival upon malaria parasite challenge.

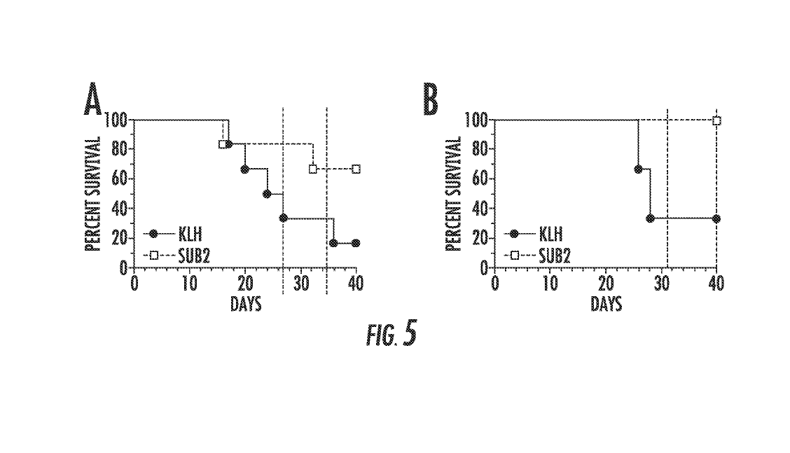

To measure survival, KLH- and SUB2-immunized (IFA or CFA) mice were monitored for forty days following P. berghei challenge. SUB2-immunized mice showed increased survival over control KLH-immunized mice for both the IFA (FIG. 5A) and CFA immunization protocols (FIG. 5B). On average, Sub2-immunized mice survived for more than one week longer than KLH control mice (summarized in Table 2). Taken together, these results suggest that the attenuated malaria parasite growth seen in SUB2-immunized mice also translates to an increased survival following P. berghei challenge.

### Example 7

SUB2 immune sera does not interfere with ookinete invasion in passively immunized mice.

One previous study has reported that SUB2 is expressed by ookinetes and is presumably secreted into the cytoplasm of ookinete-invaded cells as the parasite traverses the midgut epithelium Immunofluorescence staining identified SUB2 protein aggregates in close proximity to the actin cytoskeleton that suggest SUB2 may play an important role in cytoskeleton modifications during the process of ookinete invasion.

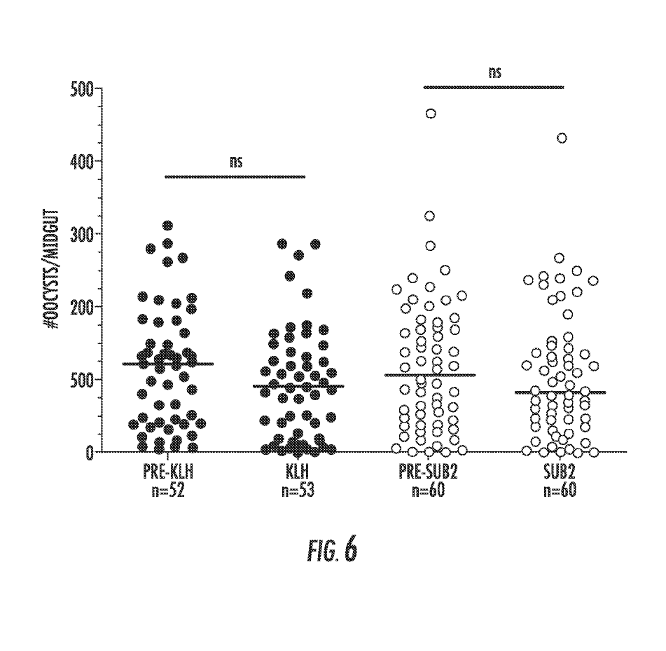

To address the role of SUB2 in ookinete midgut invasion, and the potential role that SUB2 immune sera could also inhibit ookinete invasion, passive immunization assays were performed to determine the effects on parasite development in the mosquito. As expected, passive immunization with the control KLH immune sera did not significantly alter Plasmodium oocyst numbers (FIG. 6). Similarly, passive immunization with SUB2 immune sera did not significantly alter oocyst numbers (FIG. 6), suggesting that SUB2 may either not be required for ookinete invasion of the mosquito midgut or that our immune sera was present in sub-optimal levels needed to inhibit ookinete invasion. These research questions highlight the need for further investigation into the role of SUB2 during the mosquito stages of Plasmodium development.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An immunogenic composition comprising one or more subtilisin-like protease 2 antigens from Plasmodium and a pharmaceutically acceptable carrier.

2. The immunogenic composition of claim 1, wherein the subtilisin-like protease 2 antigens are selected from P. berghei, P. falciparum, P. vivax, P. knowlesi, and P. yoelli.

3. The immunogenic composition of claim 1, wherein the subtilisin-like protease antigen has an amino acid sequence selected from the group consisting of SEQ ID NOS: 1-12.

4. The immunogenic composition of claim 3, further comprising an adjuvant.

5. The immunogenic composition claim 3, further comprising at least one additional therapeutic agent.

6. A vector comprising one or more subtilisin-like protease 2 antigens from Plasmodium.

7. The vector of claim 6, wherein the subtilisin-like protease 2 antigens are selected from P. berghei, P. falciparum, P. vivax, P. knowlesi, and P. yoelli.

8. The vector of claim 6, wherein the subtilisin-like protease has an amino acid sequence selected from the group consisting of SEQ ID NOS: 1-12.

9. The vector of claim 7, wherein the subtilisin-like protease 2 antigens are codon harmonized.

10. A cell expressing the vector of claim 8.

11. A method for blocking transmission of a Plasmodium infection in a subject comprising administering to the subject an effective amount of the immunogenic composition of claim 1.

12. The method of claim 11, wherein the subtilisin-like protease 2 antigens are selected from P. berghei, P. falciparum, P. vivax, P. knowlesi, and P. yoelli.

13. The method of claim 11, wherein the subtilisin-like protease antigen has an amino acid sequence selected from the group consisting of SEQ ID NOS: 1-12.

14. The method of claim 11, further comprising an adjuvant.

15. The method of claim 14, further comprising an additional therapeutic agent.

16. A method for immunizing a subject against Plasmodium infection comprising administering to a subject an effective amount of the immunogenic composition of claim 3.

17. The method of claim 16, wherein the subtilisin-like protease 2 antigens are selected from P. berghei, P. falciparum, P. vivax, P. knowlesi, and P. yoelli.

18. The method of claim 16, wherein the subtilisin-like protease antigen has an amino acid sequence selected from the group consisting of SEQ ID NOS: 1-12.

19. The method of claims 18, further comprising an adjuvant.

20. The use method of claim 18, further comprising an additional therapeutic agent.

21. A method for treating or preventing malaria in a subject comprising administering to a subject an effective amount of the immunogenic composition of claim 1.

22. The method of claim 21, wherein the subtilisin-like protease 2 antigens are selected from P. berghei, P. falciparum, P. vivax, P. knowlesi, and P. yoelli.

23. The method of claim 22, wherein the subtilisin-like protease antigen has an amino acid sequence selected from the group consisting of SEQ ID NOS: 1-12.

24. The method of claim 23, further comprising an adjuvant.

25. The method of claim 23, further comprising an additional therapeutic agent.


SEQUENCE LISTING (16 sequences)

SEQ ID NO: 1 - protein, 13 aa, Plasmodium berghei
KYSDRYEMTDEFL

SEQ ID NO: 2 - protein, 13 aa, Plasmodium falciparum
EYNEKYEMTQDFY

SEQ ID NO: 3 - protein, 13 aa, Plasmodium vivax
EYSEQTEMTQDFY

SEQ ID NO: 4 - protein, 13 aa, Plasmodium knowlesi
EYSEQYEMTEDFY

SEQ ID NO: 5 - protein, 13 aa, Plasmodium yoelii
KYSDRYEMTDDFF

SEQ ID NO: 6 - protein, 13 aa, Plasmodium berghei
RTSIKIVSKDKKT

SEQ ID NO: 7 - protein, 13 aa, Plasmodium falciparum
RTSIKIISTKKRT

SEQ ID NO: 8 - protein, 13 aa, Plasmodium vivax
RTSIKVISRRRRT

SEQ ID NO: 9 - protein, 13 aa, Plasmodium knowlesi
RTAIKIISRRRRT

SEQ ID NO: 10 - protein, 13 aa, Plasmodium yoelii
RTSIKIVSKDKKT

SEQ ID NO: 11 - protein, 15 aa, Artificial Sequence
  synthetic sequence
KYSDRYEMTDELFDC

SEQ ID NO: 12 - protein, 15 aa, Artificial Sequence
  synthetic sequence
CRTSIKIVSKDKKTI

SEQ ID NO: 13 - DNA, 40 bp, Artificial Sequence
  synthetic sequence
ctccatggcgaataattcaaatgcatttttgagtgtagac

SEQ ID NO: 14 - DNA, 38 bp, Artificial Sequence
  synthetic sequence
acggatccgttatcatgctcatataaattatataaagc

SEQ ID NO: 15 - DNA, 40 bp, Artificial Sequence
  synthetic sequence
atccatggcgaataataaaaaaattttgttaaatgttgat

SEQ ID NO: 16 - DNA, 38 bp, Artificial Sequence
  synthetic sequence
acggatccactatcatattcatacaaattatataaggc